(19) United States
(12) Patent Application Publication
(10) Pub. No.: US 2008/0096853 A1
(43) Pub. Date: Apr. 24, 2008

(54) Edible Compositions Comprising A Primary Lipid, A Co-Lipid, A Lipohilic Physiologically Active Ingredient And Water, And Their Preparation

(76) Inventors: Johannes Matheus M van Amelsvoort, Vlaardingen (NL); Gustaaf Sevaas M J E Duchateau, Vlaardingen (NL); Sergey Michailovich Melnikov, At Vlaardingen (NL); Ingrid Winter, Vlaardingen (NL)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(21) Appl. No.: 11/794,596

(22) PCT Filed: Dec. 7, 2005
(86) PCT No.: PCT/EP05/13106

§ 371 Date: Jun. 29, 2007

(30) Foreign Application Priority Data
Dec. 30, 2004 (GB) 0428515.1

(51) Int. Cl. A23L 1/39; A23D 7/00; A61K 47/12; A61K 31/56; A23L 2/00

(52) U.S. Cl. 514/182; 426/601; 426/590; 426/589; 514/784

(57) ABSTRACT

A composition comprising: a) a primary lipid component obtainable as the reaction product of one or more carboxylic acids with at least one of glycerol and propylene glycol; b) a co-lipid component selected from at least one of phosphorus-containing lipids and hydroxylated carboxylic acid esters of mono- and di-glycerides; c) lipophilic physiologically active ingredient; and d) water. The composition may be used to improve the bioavailability of the lipophilic physiologically active ingredient in food or beverage products. The physiologically active ingredient may be selected from carotenoids, fat-soluble vitamins, phytosterols, phytostanols and combinations thereof.

### FIELD OF THE INVENTION

The present invention relates to edible compositions which contain a lipophilic physiologically active ingredient, in particular a fat-soluble nutrient (FSN).

### BACKGROUND OF THE INVENTION

Numerous factors determine the bioavailability of a lipophilic physiologically active ingredient, such as a nutrient selected from carotenoids, vitamins A, D and E. These physiologically active ingredients may typically be coming from natural fruit, vegetable or plant sources. As a rule, the bioavailability of lipophilic, especially fat-soluble, physiologically active ingredients from raw plants is fairly low. Homogenization of fruit, vegetables or plant parts leads to the disruption of cell membranes and improvement of the bioavailability of said active ingredients. Gentle cooking (e.g., steaming, but not extensive boiling) also improves bioavailability thereof. Finally, it is well established that the bioavailability of lipophilic, especially fat-soluble, physiologically active ingredients can be significantly improved if vegetables, fruits and plant parts are consumed together with a sensible amount of dietary fat.

When free lipophilic physiologically active ingredients are to be introduced into the diet as supplements, the bioavailability issue is the major one, since formulation of these ingredients in a free form in foods results in extremely low bioavailability. In an attempt to improve bioavailability, it is known to formulate said ingredients with oil (eg, in the form of emulsions and microemulsions), as microcrystals entrapped and stabilized in a food-grade matrix, or in natural lipid (e.g., phospholipid) aggregates, such as liposomes. Such techniques are known to have a positive effect on the bioavailability of said lipophilic physiologically active ingredients.

In P. Borel, Clin. Chem. Lab. Med. (2003) 41, 979-994, it is described that the presence of fat and structure of food matrix affect carotenoid bioavailability from foods.

E. Li, P. Tso., Curr. Opin. Lipid. (2003) 14, 241-247, describes how the absorption of vitamin A in the body is closely coupled with fat absorption.

V. Tyssandier et al., Am. J. Physiol. (2003) 284, G913-G923, discloses that the high bioavailability of carotenoids from vegetables is provided if digestible fat is present in the diet. The stomach initiates the transfer of carotenoids from the vegetable matrix to the fat phase of the meal. In the intestine, the transport of carotenoids from digested fat phase to dietary micelles occurs faster than from vegetable matrix, which is low in fat.

K. H. van het H of et al., J. Nutr. (2002) 132, 503-506 discloses that the type of food matrix is a major factor that affects the bioavailability of carotenoids. Dietary fat (though in low quantities) is important for improved carotenoids absorption from natural sources.

J. D. Ribaya-Mercado, Nutr. Rew. (2002) 60, 104-110 discloses that dietary fat facilitates the absorption of carotenoids.

Other efforts have been made for preparing edible formulations with the incorporated lipophilic physiologically active ingredient that can be dispersed in an aqueous phase. For example, U.S. Pat. No. 6,426,078 describes a microemulsion of the oil-in-water type, which contains at least one polyglycerol ester as an emulsifier and at least one lipophilic substance as an internal phase. The emulsifier contains glycerol monofatty acid ester and the lipophilic substance is one from the group carotenoids, especially β-carotene, vitamins A, D, E and K and their derivatives and polyunsaturated fatty acids.

EP-A-0 818 225 describes a process for the extraction of lycopene and extracts containing it. Pure lycopene or of the lipophilic extracts containing it are prepared from whole fruits or Lycopersicum esculentum and similar species obtainable as by-products of food industry processes. The partially dehydrated fresh material is extracted with aliphatic or aromatic hydrocarbons or water-immiscible solvents in the presence of phospholipids as surfactants and stabilizing agents and the extracts are concentrated to an oil or fractionated to the desired lycopene concentration.

U.S. Pat. No. 6,261,622 relates to water-dispersible carotenoid pigment preparation. A water-dispersible carotenoid pigment preparation, which can be added to various aqueous compositions with retaining dispersion stability excellent in a wide temperature range is prepared with the soybean extract fiber as an effective ingredient.

EP-A-0 602 137 relates to a carotenoid composition derived from all natural sources, in particular the composition is an emulsion or dried product and a process for producing an all natural carotenoid composition.

U.S. Pat. No. 3,734,745 discloses formation of a dry mix comprising a gelatin portion, sugar, a fat portion where the fat is preferably plated on sugar or combined with sugar in the form of chips, a fat emulsifier, a fat soluble edible coloring agent and a water soluble coloring agent.

JP-A-08/259,829 describes a water-soluble lycopene preparation capable of blending with various aqueous compositions simply without using a surfactant and having sufficient solubility and stability.

JP-A-05/284232 covers the addition of viscous polysaccharide such as pectin, or xanthane gum together with a carotenoid pigment, a method of using a water-dispersible carotenoid pigment powdery composition comprising a carotenoid pigment having a particle diameter of less than 0.1 μm and sodium laurylsulfate.

Other references in this field include a method of dispersing a pulverized carotenoid pigment in an aqueous composition (Japanese Patent Application Laid-open No. 7-90188), a method of incorporating a carotenoid pigment, particularly β-carotene, in cyclodextrin and dispersing it in an aqueous composition (Japanese Patent Application Laid-open No. 62-267261) and a method of incorporating lycopene, one of carotenoid pigments, in γ-cyclodextrin, and adding the resulting inclusion compound in an aqueous composition together with gluten and/or ascorbic acid (Japanese Patent Application Laid-open No. 8-259829).

According to U.S. Pat. No. 6,267,963 plant sterols, plant stanols, plant sterol esters and other non-toxic sterols are co-crystallized with emulsifiers to form a plant sterol/emulsifier complex or plant stanol/emulsifier complex which can be incorporated into full-fat, reduced-fat, low-fat, fat-free and triglyceride-free food products. Plant sterols and plant stanols can be co-crystallized with emulsifiers to produce a blend which has a melting temperature significantly lower than the melting temperature of the plant sterol or plant stanol. Such complexes can be used to incorporate relatively high levels of such sterols/stanols in food products without the adverse organoleptic effect normally associated with the use of such plant sterols and plant stanols.

WO-A-2002/064110 describes a method for encapsulating in liposomes substantially water immiscible carotenoids.

U.S. Pat. No. 6,287,615 describes a method of coloring a food preparation with a carotenoid by admixing an effective amount of a carotenoid preparation with the food preparation, wherein the carotenoid preparation is an aqueous composition comprising of from 3.6 to 10% by weight of one or more carotenoids in the form of micelles.

### DEFINITION OF THE INVENTION

In a first aspect, the present invention relates to a composition that may suitably be used for incorporation of a lipophilic physiologically active ingredient into a food product, the composition comprising:
(a) a primary lipid component obtainable as the reaction product of one or more carboxylic acids with at least one of glycerol and propylene glycol;
(b) a co-lipid component selected from at least one of phosphorus-containing lipids and hydroxylated carboxylic acid esters of mono- and di-glycerides;
(c) lipophilic physiologically active ingredient; and
(d) water.

A composition according to the present invention preferably has a pH of from 3 to 8.

Compositions according to the present invention preferably comprise a lipid phase which is a crystalline or mesophase of the primary and co-lipid components. The lipophilic physiologically active ingredient should be present in the composition as a molecular dispersion, rather than crystalline or solid form, in order to ensure its bioavailability.

Preferably, the primary lipid component is obtainable as the reaction product (e.g. an ester) of at least one of lactic acid, citric acid, malic acid, maleic acid and tartaric acid with at least one of glycerol and propylene glycol.

As defined herein before, the co-lipid component is selected from at least one of phosphorus-containing lipids and hydroxylated carboxylic acid esters of mono- and di-glycerides.

Preferred phosphorus-containing lipids may be selected from lecithin, phospholipids and lysophospholipids and mixtures thereof. The co-lipid may additionally or alternatively comprise one or more hydroxylated carboxylic acid esters of mono- and di-glycerides selected from CITREM and DATEM.

Some co-lipid components can be of the same chemical class as that defined for the primary lipid component. However, it is a requirement of the present invention that in any actual composition of the invention, the co-lipid component must comprise a chemical entity different from any which is used as all or part of the primary lipid component.

The term ‘lipophilic physiologically active ingredient’, as used herein, refers to any lipophilic compound having any kind of physiological activity upon enteral administration to a human or animal body, in particular any kind of activity that is beneficial for the health of said human or animal body, either because it is essential for normal development, growth and/or functioning of a human or animal body, or because it may help to prevent or remedy any kind of abnormal physiological state in a human or animal body. Active ingredients that are essential for normal development, growth and/or functioning may for example include carotenoids and vitamins A, D and E. Suitable examples of active ingredients that, though not being essential for normal physiological functioning, have some kind of beneficial activity include, for instance, certain plant sterols that are known to have beneficial effects in subjects suffering from overweight or hypercholesterolemia. The term ‘lipophilic’ as used herein refers to the ability of a substance to be dissolved in fats and/or organic solvents. Lipophilic substances typically are poorly soluble in aqueous systems but are soluble in fats and/or organic solvents. The partition coefficient of a molecule that is observed between water and n-octanol (at 20° C.), referred to as logP or logK_{ow}, has been adopted as the standard measure of lipophilicity. According to the invention the present lipophilic physiologically active ingredients typically have a logP value of at least 3, preferably at least 3.5.

According to a preferred embodiment of the present invention, the lipophilic physiologically active ingredient is a fat-soluble nutrient, even more preferably a fat-soluble nutrient selected from carotenoids, fat-soluble vitamins, phytosterols and derivatives thereof, phytostanols and derivatives thereof, and mixtures thereof. Preferred carotenoids and fat-soluble vitamins include β-carotene, lycopene, lutein, zeaxanthine, vitamin A, vitamin D, vitamin E and vitamin K and derivatives thereof. Mixtures of any of these may be used. Preferred carotenoids and fat-soluble vitamins include β-carotene, lycopene, lutein, zeaxanthine, vitamin A, vitamin D, vitamin E and vitamin K and derivatives thereof. Mixtures of any of these may be used. Phytosterols and phytostanols, although relatively insoluble in some fats, are still within the class of fat-soluble nutrients. However, solubilised analogs of these which may be used are their fatty acid esters.

In the present invention the lipophilic physiologically active ingredient is preferably a compound that can be obtained from a natural plant, fruit or vegetable source, e.g. a vitamin or a plant sterol, also referred to as phytosterol.

According to a particularly preferred embodiment, the lipophilic physiologically active ingredient in accordance with the present invention is a steroidal glycoside extracted, e.g. a steroidal glycoside extracted from plants of the genus Trichocaulon or of the genus Hoodia said steroidal glycoside having appetite suppressant activity, as described in the international patent application WO 98/46243, which is incorporated herein by reference.

In a particularly advantageous embodiment of the invention, the lipophilic physiologically active ingredient is selected from the group consisting of appetite suppressing steroidal glycosides that can be extracted from plants of the genus Trichocaulon or of the genus Hoodia, derivatives, salts and analogs of said steroidal glycosides and mixtures thereof.

According to another preferred embodiment, the lipophilic physiologically active ingredient is a steroidal compound having the structural formula (I) or a salt or ester thereof: 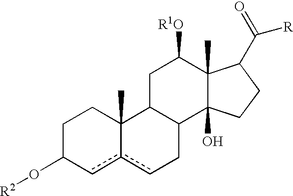
wherein
R=alkyl;
R¹=H, alkyl, tiglyol, benzoyl or any other organic acid group;
R²=H or one or more 6-deoxy carbohydrates, or glucose molecules, or combinations thereof; and wherein the broken lines indicate the optional presence of a further bond between carbonatoms C4 and C5 or between carbonatoms C5 and C6.

According to another equally preferred embodiment, the present lipophilic physiologically active ingredient is a plant extract from plants of the group comprising the genus Trichocaulon and the genus Hoodia, said extract having appetite suppressant activity. More preferably said plant extract is selected from the group consisting of appetite suppressant Trichocaulon piliferum extracts, appetite suppressant Trichocaulon officinale extracts, appetite suppressant Hoodia currorii extracts, appetite suppressant Hoodia gordonii extracts, appetite suppressant Hoodia lugardii extracts and mixtures thereof.

Preferably, the total amount of primary lipid components plus co-lipid component is from 0.01% to 20% by weight of the total composition. Preferably, the weight ratio of the primary lipid component to the co-lipid component is from 1:10 to 100:1, more preferably from 1:10 to 10:1.

Compositions according to the present invention preferably also contain from 75% to 99.9% by weight of the water, more preferably from 80% to 95% by weight. In a preferred embodiment the compositions of the present invention are water-continuous.

The total amount of lipophilic physiologically active ingredient in any composition according to the invention preferably exceeds 0.0001% by weight. Even more preferably the amount of lipophylic physiologically active ingredient is from 0.001% to 5% by weight of the composition, most preferably from 0.01 to 2.5% by weight.

Compositions according to first aspects of the present invention may be incorporated in a wide range of food products, for example, beverages, dressings, soups, sauces, dips, spreads or fillings. The amount of the composition to be incorporated in said food products is typically sufficient to provide from 0.0001%, eg from 0.001%, in particular from 0.01 to 25%, preferably from 0.1% to 10% by weight of total lipophilic physiologically active ingredient in the final food product.

Possible amounts of incorporation also include those sufficient to provide from 0.5% or from 1% and up to 5% or up to 2% or even up to 1% by weight of said active ingredient in the food product.

Typically, food products which incorporate compositions according to the present invention contain at least one component other than the composition itself. A method according to the invention for making such a food product will comprise admixture in any order, of the composition according to the first aspect of the invention and the one or more other components.

Low-fat product is to be understood herein as to mean any food product containing less than 1.5% of fat, excluding the amount of lipids that may be used as colipids for the preparation of lipid phase of the first aspect of this invention.

Other aspects of the invention relate to a method of improving the bioavailability of a lipophilic physiologically active ingredient, said method comprising incorporating the lipophilic physiologically active ingredient, as defined herein before, in a composition comprising:
(a) the primary lipid component obtainable as the reaction product of one or more carboxylic acids with at least one of glycerol and propylene glycol;
(b) the co-lipid component selected from at least one of phosphorus-containing lipids and hydroxylated carboxylic acid esters of mono- and di-glycerides; and
(c) water; said composition preferably being water continuous; and to the use of the said composition for improving the bioavailability, in particular the oral bioavailability, of a lipophilic physiologically active ingredient, as defined herein before.

The present invention will now be explained in more detail by way of the following non-limiting examples.

### EXAMPLE 1

6 mg of β-carotene and 2 g of lactic fatty acid ester+glycerol/propylene glycol reaction product (LFEGPG) were dissolved at 55° C. in 10 ml of ethanol. This solution was added dropwise to 200 ml of intensely agitated Tris buffer (0.1 g). After 15 minutes of stirring, the solution was cooled down and the pH adjusted to 4.0 with 1M HCl. The precipitate was then filtrated and LFEGPG lipid phase containing molecularly-dispersed β-carotene was used further in this experiment.

The precipitate was added to the USP24 vessel (dissolution test type II) loaded with 900 ml of bile extract solution in aqueous buffer at 37° C. As a control, 6 mg of free crystalline β-carotene was added to the same buffered bile solution in USP24. After stirring in USP24 for 60 min, it was surprisingly found that while the solubility of free β-carotene was almost 0, solubility of β-carotene from LFEGPG lipid phase in the bile solution was as high as 26% from the initial amount (6 mg).

### EXAMPLE 2

Carrot juice with free LFEGPG and lecithin, as well as mixed LFEGPG-lecithin lipid phases with different LFEGPG-to-lecithin ratios were prepared as described below:

LFEGPG Addition to Juice:

The amount of molten LFEGPG (55° C.) is added to 315 mL of 50° C. pre-warmed carrot juice (97% pure fresh carrot juice, Zonnatura B.V Zoetermeer, Netherlands) in an Erlenmeyer. LFEGPG is slowly poured to a juice being stirred. The juice is then left with an intense stirring in a 50° C. bath, for 20 minutes. The juice is then poured into a beaker, for undergoing a 1-minute homogenisation. This is done using a Silverson L4RT-A homogenizer at 5000 rpm (with small window screen). The juice is then relocated into an Erlenmeyer placed in a 50° C. water bath with stirring, for 10 minutes. The Erlenmeyer is then left at room temperature and with stirring for about 2 hours.

The 315 ml of juice is then immediately mixed with buffer, bile and pancreatine in a USP Type II dissolution-apparatus to mimic gastrointestinal conditions.

Lecithin Addition to Juice:

The method is exactly the same as previously described above. Nevertheless, the lecithin is added cold and in a solid state to juice. The lecithin used is phosphatidylcholine from Degussa-Bioactives (Epikuron 200, 92% unsaturated phosphatidylcholine, extracted from soy).
LFEGPG/Lecithin Mixture Addition to Juice:

The protocol is similar to the two described above. First lecithin is added to the 50° C. juice (like described), and 2 minutes later the molten LFEGPG. The juice is then homogenised like described.

The amount of lecithin is added to pre-warmed (50° C.), and continuously stirred carrot juice (97% pure fresh carrot juice, Zonnatura BV, Zoetermeer, Netherlands). Subsequently, the Durlac is slowly added to the juice being stirred at 50 C. The juice is then left with an intense stirring in a 50 C waterbath for 20 minutes. The juice is then poured into a beaker for undergoing a 1-minute homogenization. This homogenization is done using a Silverson L4RT-A homogenizer at 5000 rpm (with small window screen). The juice is relocated into an Erlenmeyer placed in a 50 C water bath with stirring for 10 minutes. The Erlenmeyer is then left at room temperature and with stirring for about 2 hours. The 315 mL of juice are then immediately mixed with buffer, bile and pancreatine in the USP type II dissolution apparatus to mimic the gastro-intestinal conditions.

Surprisingly, the in vitro bioaccessibility of β-carotene, expressed as the percentage of total carotenoids solubilized in biliary solution, is the highest for the lecithin-LFEGPG mixed lipid phase (see Table 1 and Table 2). It is higher than values for free lecithin or free LFEGPG, and the optimal ratio between lecithin and LFEGPG is close to 3:1, respectively.

TABLE 1
Percentage of carotenoids transferred from carrot
juice to the bioaccessible, micellar space in 60 min
during the USP dissolution test, using NPL/colipid
mictures.

| composition (%) | |
|---|---|
| NPL(LFEGPG)/co- | |
| lipid (lec) | % in micellar space |
| mixes | (mean ± SD) |
| 1 + 0 | 11.5 ± 1.2 |
| 0.75 + 0.25 | 20.7 ± 0.1 |
| 0.50 + 0.50 | 31.0 ± 0.2 |
| 0.25 + 0.75 | 33.7 ± 0.3 |
| 0 + 1 | 24.0 ± 0.5 |

TABLE 2
Percentage of carotenoids transferred from carrot
juice to the bioaccessible, micellar space in 60 min
during the USP dissolution test, using colipids
alone.

| composition (%) | % in micellar space |
|---|---|
| co-lipid (lec) | (mean ± SD) |
| 0 | 15.6 ± 0.3 |
| 0.25 | 21.9 ± 0.1 |
| 0.50 | 24.5 ± 0.6 |
| 0.75 | 24.4 ± 0.4 |
| 1 | 24.0 ± 0.5 |

### EXAMPLE 3

Homogenization efficiency has an effect on the in vitro bioaccessibility of carotenoids. In this example one dispersion was prepared with an Ultra Turrax T25 (IKA Labortechnik), another with a Silverson homogenizer, both samples were homogenized for ? min. FIG. 3 represents the effect of homogenization efficiency on the solubilization of b-carotene entrapped into the 3:1 lecithin-LFEGPG mixed lipid phase. Silverson provides smaller lipid phase particle size and, additionally, faster hydration of lipid.

### EXAMPLE 4

This example shows that in the mixed lecithin-LFEGPG systems, the order of the lipid addition to the juice can be of importance. If lecithin is added before LFEGPG, as described in the Example 1, then the solubilization of b-carotene by biliary micelles from lipid phases is greater than that in case of LFEGPG addition before lecithin. If mixed LFEGPG-lecithin film, prepared by dissolving LFEGPG and lecithin in organic solvent and its consequent evaporation, is added to the biliary micelles, then the recovery of b-carotene is the lowest (FIG. 4).

### EXAMPLE 5

The standard protocol described for mixing lecithin and LFEGPG with juice is composed of several steps. First the lecithin is added to a juice equilibrated at 50° C. Then molten LFEGPG is added and a Silverson homogenisation is performed for 20 minutes. The homogenised juice is then left for 10 minutes to allow some carotenoid exchanges between the juice matrix and the lecithin-LFEGPG lipid phase. These 10 minutes have been extended to 24 hours to investigate any time effect on this step. A nitrogen flow was also used to avoid any oxidation phenomenon during this time. The results are presented in. FIG. 5. It can be seen that, taking the standard deviation into the account, the extension of the extraction time from 10 min to 24 hrs does not lead to the increase in β-carotene solubilization by the biliary micelles in vitro.

### EXAMPLE 6

### Incorporation of β-Carotene in Model Systems

(1) A. 1 g LFEGPG and 2.4 mg pure β-carotene were added to 100 ml water, followed by magnet stirring for 1 h at 50° C. 1.7 mg β-carotene/g dry LFEGPG were detected by UV-Vis spectrophotometry after centrifugation and extraction of β-carotene from the collected lipid phase by hexane.
B. 1 g LFEGPG and 2.4 mg pure β-carotene were melted together and added dropwise to 100 ml water, followed by magnet stirring for 20 min at 50° C. Ultra Turrax was applied to homogenize the sample, then the sample was magnet stirred for another 10 min at 50° C. 2.2 mg β-carotene recovered by UV-Vis spectrophotometry after centrifugation and extraction by hexane.
(2) A. 3 g LFEGPG and 15 mg β-carotene were melted together at 65° C. in a water bath and added dropwise to 300 ml 65° C. warm water (water bath), while homogenizing with Silverson. B. 0.3 g Span 60 (sorbitan monostearate) were dissolved in 300 ml water at 65° C. A melt of 3 g LFEGPG and 15 mg β-carotene (65° C.) was homogenized with Silverson as described under A.
Microscope: Significantly reduced number of free β-carotene crystals in sample B.
(3) 0.9 g LFEGPG, 5 mg β-carotene, and 0.1 g of one of the following colipids CITREM (citric acid esters of mono- and diglycerides), Span 60 (sorbitan monostearate) or Epikuron 100 G (deoiled granulated soybean lecithin), were dissolved at 55° C. in 10 ml of ethanol. This solution was added dropwise to 200 ml of intensely agitated Tris buffer (0.1 g). After 15 minutes of stirring, the solution was cooled down and the pH adjusted to 4.0 with 1M HCl. Polymorphic or mesomorphic lipid phases containing molecularly dispersed β-carotene were derived (judged from appearance under microscope, however also a few free β-carotene crystals were detected, thus not completely molecularly dispersed).
(4) As the solubility of β-carotene in ethanol is limited, complete molecular dispersion of β-carotene was achieved by the use of non food-grade solvents such as chloroform. 1 g LFEGPG and 3 mg β-carotene were dissolved in 5 ml chloroform and added to 100 ml water under intense magnet stirring at 60° C. The solution was kept at 60° C. for 30 min. The lipid phase was investigated under the optical light microscope, and no β-carotene crystals could be detected.
(5) 0.2 g of LFEGPG and 2 mg β-carotene as well as 0.2 g a LFEGPG/Datem (diacetyl tartaric acid esters of mono- and diglycerides) mixture (93/7, by wt) and 2 mg β-carotene were dissolved in chloroform. The lipid mixtures were dried under a stream of nitrogen and kept under vacuum over night to remove the chloroform completely. The dried lipid films were hydrated with a phosphated buffered solution (20 mM Na-phosphate, 130 mM NaCl, pH 6.1) for 30 min at 60° C. Micrographs showed that part of the β-carotene is molecularly dispersed by applying this method and that the admixing of Datem as a colipids decreases the amount of free β-carotene crystals.
(6) The same preparation method as described under (6) was applied to a mixture of 80% LFEGPG and 20% Epikuron 200 (purified soybean lecithin with min. 92% phosphatidylcholine) and varying amounts of β-carotene (4 to 6 mg β-carotene/g lipid). Micrographs showed no free β-carotene crystals in the sample containing 4 mg β-carotene, a few free β-carotene crystals in the sample containing 5 mg β-carotene, but a comparatively large amount of free crystals in the sample containing 6 mg β-carotene.

### EXAMPLE 7

### In Situ Incorporation of Carotenoids into LFEGPG

1) 1 g solid LFEGPG was added to 100 ml cold carrot juice (97% pure fresh carrot juice, Zonnatura B.V Zoetermeer, Netherlands). The mixture was warmed up to 50° C. and kept at this temperature for 30 min with magnetic stirring. Homogenization was performed with a Silverson homogenizer at ˜5500 rpm followed by another hydration step for 5 min at 50° C.
The LFEGPG phase was filtrated, collected and dissolved in hexane. The β-carotene content was determined spectrophotometrically (absorbance at 448 nm) to be 0.68 mg β-carotene/g dry lipid phase (corrected for a water content of the hydrated LFEGPG phase of 30%).
(2) 2 g LFEGPG were melted at 50° C. and added dropwise to 100 ml carrot juice kept at 50° C. Magnet stirring was performed for 1 hour at 50° C., then the solution was homogenized with a Silverson homogenizer (4500 rpm for 1 minute) and magnet stirred at 50° C. for another 1 hour. The LFEGPG phase was collected by filtration and dissolved in hexane. The β-carotene content was spectrophotometrically determined (absorbance at 448 nm) to be 1.5 mg β-carotene/g dry LFEGPG. The same procedure was reproduced in a matrix of tomato juice and tomato paste and led to the similar result, which shows that this method is not exclusive to a carrot juice, but can also be used in other natural FSN-rich formats.
(3) 1 g LFEGPG was melted at 50° C. and added dropwise to 100 ml carrot juice kept at 50° C. Magnet stirring was performed for 20 min at 50° C., Silverson (4500 rpm, 1 min.), and magnet stirred at 50° C. for another 10 min at 50° C. The LFEGPG phase was collected by filtration and dissolved in hexane. The β-carotene content was spectrophotometrically determined (absorbance at 448 nm) to be 2.0 mg β-carotene/g dry LFEGPG.

### EXAMPLE 8

### Incorporation of an Extract Comprising Appetite Suppressing Steroidal Glycosides

0.1 g Hoodia extract (PYM 50987) was mixed with 0.4 g of a lipid mixture comprising of 90% Durlac 300 and 10% Lactem and dissolved in 3 ml absolute ethanol at 80° C. The hot solution was added dropwise into 100 ml of a 60° C. warm aqueous solution (0.1 M Na₂HPO₄*2H₂O, pH=9), which was intensely stirred with a magnet stirrer. The dispersion was slowly cooled down to room temperature, and the pH was reduced to about neutral by addition of HCl. The dispersion was filtrated with a paper filter, and the slurry was air-dried and re-dispersed in water by high-shear mixing with an Ultra-Turrax mixer. Upon energy input (e.g. by sonication for 20 min.), mesophases were formed from this re-dispersion (proven with polarized light microscopy: appearance of typical maltese crosses with crossed polarizers).

The maximum amount of Hoodia actives, which can be incorporated into the bilayer of a lipid mesophase, was investigated with pure model substances by differential scanning calorimetry. Samples were prepared from 1,2-Dimyristoyl-sn-Glycero-3-Phosphocholine (DPPC, Sigma P4329), which self-assembles into bilayers upon dispersion in polar solutions, and the Hoodia extract PYM50027 (containing ˜73% actives). The typical peak observed for DPPC at ˜42° C. (for the conditions: pH 7.4, 20 mM PBS, 150 mM NaCl) for the transition from the gel phase to the liquid-crystalline phase (both are “mesophases”) is distinctively changing upon the addition of Hoodia extract. From the maximum amount of Hoodia extract, which still induced a significant change of the peak shape observed by DSC, it was estimated that a maximum amount of 25 mol % Hoodia actives can be incorporated into the DPPC bilayer. Similar values can be expected for mesophases formed by other lipids.

1. A lipid phase containing composition comprising:—
(a) a primary lipid component obtainable as the reaction product of one or more carboxylic acids with glycerol and propylene glycol;
(b) a co-lipid component selected from at least one of phosphorus-containing lipids and hydroxylated carboxylic acid esters of mono- and di-glycerides;
(c) lipophilic physiologically active ingredient; and
(d) water;
wherein the lipid phase is a crystalline or mesophase of the primary and co-lipid components.

2. A composition according to claim 1, wherein the primary lipid component is obtainable as the reaction product of at least one of lactic acid, citric acid, malic acid, maleic acid and tartaric acid with glycerol and propylene glycol.

3. A composition according to claim 1, comprising a phosphorus-containing lipid selected from lecithin, phospholipids and lysophospholipids and mixtures thereof.

4. A composition according to claim 1, comprising a co-lipid which comprises one or more hydroxylated carboxylic acid esters of mono- and di-glycerides.

5. A composition according to claim 1, wherein the lipophilic physiologically active ingredient is a fat soluble nutrient.

6. A composition according to claim 1, wherein the lipophilic physiologically active ingredient is selected from carotenoids, fat-soluble vitamins, phytosterols and derivatives thereof, phytostanols and derivatives thereof, and mixtures thereof.

7. A composition according to claim 1, wherein the lipophilic physiologically active ingredient is a compound having the structural formula (I) or a salt or ester thereof; 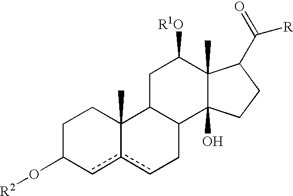
Wherein
R=alkyl;
R¹=H, alkyl, tiglyol, benzoyl or any other organic acid group;
R2=H or one or more 6-deoxy carbohydrates, or glucose molecules, or combinations thereof; and wherein the broken lines indicate the optional presence of a further bond between carbonatoms C4 and C5 or between carbonatoms C5 and C6.

8. A composition according to claim 1, comprising from 0.01% to 20% by weight of the primary lipid component plus the co-lipid component.

9. A composition according to claim 1, wherein the weight ratio of the primary lipid component to the co-lipid component is from 1:10 to 100:1.

10. A composition according to claim 1, comprising from 75% to 99% by weight of the water.

11. A composition according to claim 1, wherein the composition is water-continuous.

12. A composition according to claim 1, comprising from 0.001% to 5% by weight of the lipophilic physiologically active ingredient.

13. A food product, comprising a composition according to claim 1 in an amount sufficient to provide in the food product, from 0.001%, preferably from 0.01% to 25%, more preferably from 0.1% to 10% by weight of lipophilic physiologically active ingredient.

14. A food composition according to claim 13, in the form of a beverage, dressing, soup, sauce, dip, spread or filling.

15. A method of preparing a food product comprising a composition according to claim 1, and the one or more other ingredients, the method comprising admixture of said composition and one or more other ingredients in any order.

16. A method of improving the oral bioavailability of a lipophilic physiologically active ingredient by administering a lipid phase containing composition comprising:
(a) a primary lipid component obtainable as the reaction product of one or more carboxylic acids with glycerol and propylene glycol;
(b) a co-lipid component selected from at least one of phosphorus-containing lipids and hydroxylated carboxylic acid esters of mono- and di-glycerides; and
(c) water.
